(19) United States
(12) Patent Application Publication
(10) Pub. No.: US 2026/0325571 A1
(43) Pub. Date: Oct. 22, 2026

(54) PHARMACEUTICAL COMPOSITION CONTAINING LIPOTEICHOIC ACID FOR PREVENTING OR TREATING PERIODONTITIS

(71) Applicant: SEOUL NATIONAL UNIVERSITY R&DB FOUNDATION, Seoul (KR)

(72) Inventors: Seung Hyun HAN, Yongin-si, Gyeonggi-do (KR); Dongwook LEE, Seoul (KR); Dong Hyun PARK, Seoul (KR); Jintaek IM, Seoul (KR); Somin PARK, Seoul (KR)

(21) Appl. No.: 19/116,697

(22) PCT Filed: Sep. 6, 2023
(86) PCT No.: PCT/KR2023/013359

§ 371 Date: Mar. 28, 2025

(30) Foreign Application Priority Data
Sep. 29, 2022 (KR) 10-2022-0124161

(51) Int. Cl. A61K 31/739; A23K 20/163; A61K 35/747; A61P 1/02; A61P 29/00; A61P 31/04

(52) U.S. Cl. CPC A61K 31/739; A23K 20/163; A61K 35/747; A61P 1/02; A61P 29/00; A61P 31/04

(57) ABSTRACT

The present invention relates to a pharmaceutical composition for preventing or treating periodontitis, and an anti-inflammatory composition, the compositions comprising lipoteichoic acid or a pharmaceutically acceptable salt thereof. The present invention inhibits, even with a small amount thereof, the attachment of bacteria that cause periodontal diseases, and inhibit inflammation, and thus has high safety and few side effects while used as a biocompatible material.

### TECHNICAL FIELD

This present invention relates to a pharmaceutical composition for preventing or treating periodontitis comprising lipoteichoic acid and anti-inflammatory composition.

### BACKGROUND ART

Oral pathogenic bacteria aggregate with various other pathogenic bacteria depending on the environment, forming biofilms that confer resistance to antibiotics. Once detached from the biofilm, these bacteria can infiltrate various tissues within the human body, leading to infections and contributing to the development of systemic diseases. Thus, the biofilm of oral pathogenic bacteria plays a critical role in the onset and progression of infections and related diseases.

Periodontal disease primarily arises from bacterial infections in the oral cavity. Early-stage gum disease, known as gingivitis, is characterized by inflammation confined to the soft tissues. As the disease progresses, it can lead to periodontitis, which involves the destruction of alveolar bone.

In South Korea, periodontal disease caused by oral pathogenic bacteria affects approximately one in four individuals. In 2016, the number of patients treated for periodontal disease exceeded ten million.

A representative pathogen associated with periodontal disease is Porphyromonas gingivalis, which is predominantly detected in patients with periodontitis. This bacterium colonizes periodontal pockets between the teeth and gums, utilizing sugars and proteins from dental plaque for survival. Additionally, it secretes enzymes that degrade collagen, a key component of gingival tissue, thereby weakening the gums. P. gingivalis also exhibits invasive properties, allowing it to penetrate oral epithelial cells and contribute to the progression of periodontitis. Moreover, it has been strongly associated with various systemic diseases, including Alzheimer's disease, rheumatoid arthritis, and cancer, highlighting its broad pathological significance.

Given these implications, there is an increasing emphasis on the need for effective treatment and management of periodontal disease. Currently, the clinical treatment of periodontal disease involves the use of oral therapeutic agents, such as antibiotics or nonsteroidal anti-inflammatory drugs (NSAIDs). Prolonged antibiotic treatment can result in various side effects, including the emergence of resistant bacterial strains, alterations in the microbial flora that increase the risk of secondary opportunistic infections, and tooth discoloration. Similarly, long-term exposure to NSAIDs has been associated with gastrointestinal disorders, such as hepatotoxicity and digestive disorders.

To mitigate the risks associated with excessive pharmaceutical use in clinical practice, there is a growing demand for research on biocompatible substances that are non-toxic to humans. Such alternative therapeutic agents should not only exhibit superior safety profiles and fewer side effects compared to conventional treatments but also effectively alleviate periodontal disease.

Lipoteichoic acid (LTA), a major cell wall component derived from Gram-positive bacteria, plays a crucial role in bacterial growth, attachment, biofilm formation, and host immune system activation.

In this study, the present inventors successfully isolated and purified LTA from Lactobacillus, a well-known lactic acid bacteria, with high purity. The purified LTA was found to effectively inhibit arginine-specific gingipains, key virulence factors involved in periodontitis pathogenesis. Furthermore, LTA interacts with fimbrial proteins involved in bacterial attachment, thereby preventing bacterial attachment and alleviating infections and periodontitis. By elucidating this underlying mechanism, the present invention provides a novel therapeutic approach for the treatment of periodontitis.

### DISCLOSURE

### Technical Problem

The purpose of the present invention is to provide a pharmaceutical composition for preventing or treating periodontitis, comprising lipoteichoic acid or a pharmaceutically acceptable salt thereof.

Also, the purpose of the present invention is to provide an anti-inflammatory composition, comprising lipoteichoic acid or a pharmaceutically acceptable salt thereof.

The another purpose of the present invention is to provide an oral disease therapeutic agent, a food cleaning agent, a health functional food and a livestock feed additive comprising the composition.

However, the technical challenges intended to be addressed by the present invention are not limited to those challenges mentioned above, and other challenges not mentioned will be clearly understood by those skilled in the art from the following description.

### Technical Solution

The present inventors studied whether lipoteichoic acid could reduce the surface attachment force of periodontitis causing bacteria. As a result, it was confirmed that lipoteichoic acid directly interact with the fimbrial protein (FimA) of Porphyromonas gingivalis thereby reducing the bacterial surface attachment force leading to lowered infectivity using a mutant strain lacking for fimbrial protein (FimA) gene.

Therefore, the present invention provides a pharmaceutical composition for preventing or treating periodontitis, comprising lipoteichoic acid or a pharmaceutically acceptable salt thereof.

The term “lipoteichoic acid” in the present invention has the same meaning as “LTA” and can be used interchangeably.

The term “preventing” in the present invention means any act of inhibiting or delaying the onset of periodontitis by administering the pharmaceutical composition of the present invention, and “treating” means any act of improving or benefiting the symptoms of already-induced periodontitis by administering the pharmaceutical composition of the present invention.

The pharmaceutical composition for preventing or treating periodontitis comprising a lipoteichoic acid or a pharmaceutically acceptable salt thereof of present invention may additionally comprise an appropriate carrier, excipient or diluent according to a conventional method. Carriers, excipients, and diluents that may be included in pharmaceutical compositions comprising a compound include, but are not limited to, lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate and mineral oil.

The pharmaceutical composition for preventing or treating periodontitis comprising a lipoteichoic acid or a pharmaceutically acceptable salt thereof of the present invention can be formulated into any one dosage form selected from the group consisting of powders, pills, granules, capsules, suspensions, oral liquid preparations, emulsions, syrups, sterilized aqueous solutions, non-aqueous solutions, suspensions, lyophilized preparations and suppositories according to a conventional method. When formulating, the composition can be prepared using diluents or excipients such as fillers, bulking agents, binders, wetting agents, disintegrants, and surfactants that are commonly used. It is preferable to use suitable formulations known in the art as disclosed in a literature (Remington's Pharmaceutical Science, Mack Publishing Company, Easton PA).

The pharmaceutical composition of the present invention is administered in a pharmaceutically effective amount. The term “pharmaceutically effective amount” in the present invention means an amount sufficient to exhibit a preventive or therapeutic effect on periodontitis and an amount that does not cause side effects or serious or excessive immune responses, and the effective dosage level will vary depending on various factors including the disorder to be treated, the severity of the disorder, the activity of the specific compound, the route of administration, the duration of treatment, drugs used in combination with or concurrently with the composition comprising lipoteichoic acid, the age, weight, sex, eating habits, general health of the subject, and factors known in the pharmaceutical industry and the medical field. Various general considerations in determining a “therapeutically effective amount” are well known to those skilled in the art and are described, for example, in [Gilman et al., eds., Goodman And Gilman's: The Pharmacological Bases of Therapeutics, 8th ed., Pergamon Press, 1990] and [Remington's Pharmaceutical Sciences, 17th ed., Mack Publishing Co., Easton, Pa., 1990].

The term “administration” in the present invention means introducing a given substance into a patient by any suitable method, and is formulated for human or veterinary use and administered by various routes. The composition of the present invention may be administered by parenteral routes, such as intravascular, intravenous, intraarterial, intramuscular, or subcutaneous, and may be administered by oral, nasal, rectal, transdermal, or inhalation via an aerosol, and may be administered as a bolus or by gradual infusion.

The pharmaceutical composition of the present invention can be administered as an individual therapeutic agent or in combination with other therapeutic agents, and can be administered sequentially or simultaneously with conventional therapeutic agents. And it can be administered singly or multiple times. It is important to administer an amount that can obtain the maximum effect with the minimum amount without side effects by considering all of the above factors, and it can be easily determined by those skilled in the art.

The preferred dosage of the pharmaceutical composition according to the present invention varies depending on the patient's condition and weight, the degree of the disease, the drug form, the administration route, and the period, but can be appropriately selected by those skilled in the art. However, for a desirable effect, it is recommended to administer 0.01 mg/kg to 10 g/kg per day, preferably 1 mg/kg to 1 g/kg. The administration can be administered once a day or divided into several times. Therefore, the above dosage does not limit the scope of the present invention in any way.

The pharmaceutical composition of the present invention can comprise one or more known effective ingredients having a preventive and therapeutic effect on periodontitis together with a composition comprising lipoteichoic acid or a pharmaceutically acceptable salt thereof.

The pharmaceutical composition of the present invention can be used as a prescription drug, a hygiene product, a food and livestock feed additive, etc., to perform the function of preventing or treating periodontitis.

In another aspect, the present invention provides a method for preventing or treating periodontitis, comprising a step of administering the pharmaceutical composition to an individual other than a human that is expected to develop periodontitis or has developed periodontitis.

The term “individual” in the present invention means a living organism that can develop periodontitis, and preferably can be a higher vertebrate that can show symptoms of periodontitis, and more preferably can be livestock such as cows, pigs, sheep, goats, and dogs that can develop periodontitis, and humans that can be infected therefrom.

When the pharmaceutical composition of the present invention is administered to the individual, it can exhibit the effect of preventing or treating periodontitis by enhancing the immunity of the individual against the strain causing periodontitis. The target of administration of this pharmaceutical composition can be primarily livestock such as cows, pigs, sheep, goats, and dogs, but is not limited thereto, and can also be used for preventing or treating humans who can be infected with periodontitis by the livestock.

The lipoteichoic acid included in the pharmaceutical composition of the present invention may be derived from various gram-positive bacteria including lactic acid bacteria, which are safe beneficial bacteria that are not harmful to the human body, and may be separated from at least one selected from the group consisting of Lactobacillus rhamnosus, Lactobacillus plantarum, Lactobacillus casei, Lactobacillus acidophilus, and Lactobacillus reuteri, as a biocompatible substance, and preferably Lactobacillus rhamnosus, Lactobacillus plantarum, Lactobacillus casei and Lactobacillus reuteri, but is not limited thereto.

The periodontitis causing bacteria may be at least one selected from the group consisting of Porphyromonas gingivalis, Campylobacter gracilis, Prevotella nigrescens, Aggregatibacter actinomycetemcomitans, and Parvimonas micra. In a specific embodiment of the present invention, it was confirmed that not only gram-positive bacteria but also gram-negative bacteria can be targeted (FIG. 7).

In addition, periodontal disease pathogenic bacteria are also known to cause various systemic diseases such as Alzheimer's and cancer, so they can be used for the purpose of preventing and treating various infectious diseases through the above-described effects.

Among these, when the periodontitis causing bacteria is Porphyromonas gingivalis, the lipoteichoic acid can target the fimbrial protein FimA. In a specific embodiment of the present invention, a new signaling system was discovered in which lipoteichoic acid interacts with the fimbrial protein (FimA), one of the surface proteins of Porphyromonas gingivalis, to reduce the surface attachment and cohesion, or inflammatory response of Porphyromonas gingivalis (FIG. 4).

As shown in FIG. 1 and FIG. 2, it was confirmed that lipoteichoic acid alleviates periodontitis aggravated by Porphyromonas gingivalis in vivo and in vitro models, and also has an effect of reducing the surface attachment force of the pre-attached bacteria.

The present invention also provides an anti-inflammatory composition comprising lipoteichoic acid or a pharmaceutically acceptable salt thereof.

The lipoteichoic acid included in the anti-inflammatory composition of the present invention may be derived from various gram-positive bacteria including lactic acid bacteria, and for example, may be isolated from Lactobacillus rhamnosus, Lactobacillus plantarum, Lactobacillus casei, Lactobacillus acidophilus, and Lactobacillus reuteri, and preferably, may be isolated from Lactobacillus rhamnosus, Lactobacillus plantarum, Lactobacillus casei, and Lactobacillus reuteri, but is not limited thereto.

The inflammation may be caused by Porphyromonas gingivalis. In a specific embodiment, since periodontitis caused by Porphyromonas gingivalis is caused by a chronic inflammatory response, it was confirmed that the activity of gingipain, which controls the response, was inhibited, and when Porphyromonas gingivalis was infected with oral epithelial cells and the expression of inflammatory cytokines was confirmed, it was confirmed that when lipoteichoic acid was treated, the RNA expression levels of IL-6, IL-8, and IL-1β, which are inflammatory cytokines, were reduced (FIG. 3).

In addition, it was confirmed that this alleviates the inflammatory response by inhibiting the decomposition of MCPIP-1, which controls the inhibition of inflammatory cytokines. Therefore, the composition may alleviate the inflammatory response by inhibiting the decomposition of MCPIP-1.

In addition, the composition comprising a lipoteichoic acid of the present invention can be utilized in a wide range of industrial fields such as a periodontal disease treatment agent alternative, oral cleanser, ointment, toothpaste, oral dissolving film, etc.

Therefore, the present invention provides an oral disease treatment agent comprising the composition.

The oral disease treatment agent can include all types and formulations, including cases in which it is in the form of a medical device that can be used for oral treatment or oral hygiene, and preferably can be any one selected from the group consisting of oral cleansers, oral sprays, oral ointments, oral patches, oral artificial tooth coating agents, dental floss, and toothpaste.

When the oral disease treatment agent is a paste composition, the binder may be an organic binder such as carrageenan, various thickening cellulose derivatives, gums such as xanthan gum, tragacanth gum, polyvinyl alcohol, sodium polyacrylate, polyacrylic acid/maleic acid copolymer, synthetic polymer derivatives such as carboxyvinyl polymer and inorganic binders such as silica and laponite, used alone or in combination.

The oral disease treatment agent may additionally include sorbitol, glycerin, ethylene glycol, propylene glycol, polyether glycol, polypropylene glycol, etc. as a moisturizing agent during its preparation.

The above oral disease treatment agent may additionally include a flavoring agent or a sweetener, etc. The sweetener may be sucrose, lactose, maltose, sorbitol, xylitol, sodium cyclamate, glycerin, sodium saccharin, stevioside, aspartame, etc., and the flavoring agent may be peppermint, menthol, anethole, eugenol, limonene, citronellol, alpha-terpineol, salicylmethyl, cineole, linalool, ethyl linalool, vanillin, thymol, spearmint oil, sage oil, rosemary oil, cinnamon oil, etc., and the sweetener or flavoring agent may be used alone or in combination.

The oral disease treatment agent may include a surfactant or an additional effective ingredient used as a foaming agent alone or in combination. The surfactant used as the foaming agent may be any one selected from the group consisting of anionic surfactants, nonionic surfactants, and cationic surfactants.

Meanwhile, the oral disease treatment agent can also be used as an oral hygiene product comprising thereof, and as an active ingredient, the oral hygiene product includes oral cleaning products such as gargles, Listerine, and oral dissolving films, skin cleansing compositions such as toothpaste, oral hygiene products, soaps, hand soaps, vaginal cleanser additives, and body cleansers, kitchenware cleansing compositions for cleaning dishes, sinks, cutting boards, etc., and cosmetic compositions such as essences and creams. When added as an active ingredient to hygiene products, the product can perform a function of preventing or treating periodontitis in addition to the original function of each hygiene product.

The present invention also provides a food cleaning agent comprising the composition.

Since the food cleaning agent is a natural ingredient and is not harmful to the human body, it can be used for the purpose of cleaning or imparting antibacterial properties to various foods, comprising food washes.

The food cleaning agent may additionally contain an appropriately selected additive depending on the purpose and use thereof, and may be used by mixing it with other active ingredients, pigments, surfactants, preservatives, and other additives commonly used in cleaning agents. The food cleaning agent can be prepared by powdering, granulating, tableting or liquefying according to its intended use and application, and a conventional method can be used for packaging for productization.

The food cleaning agent can include an antibacterial composition, and the amount of the antibacterial composition can be appropriately used according to the intended use, application form, application site, application target product, etc.

Furthermore, the present invention also provides a health functional food comprising the composition. “Health functional food” means a food manufactured and processed using raw materials or ingredients having useful functionality for the human body according to Act No. 6727 on Functional Foods, and “functionality” means consuming for the purpose of obtaining a useful effect for health purposes such as regulating nutrients for the structure and function of the human body or physiological effects.

The health functional food of the present invention can be used for the purpose of preventing or treating periodontitis. When the composition comprising a lipoteichoic acid or a pharmaceutically acceptable salt thereof of the present invention is used as an additive, the composition comprising a lipoteichoic acid may be added as is or used together with other foods or food ingredients, and may be used appropriately according to a conventional method. Examples of foods to which the substance may be added include meat, processed meat, bread, chocolate, candy, snacks, confectionery, pizza, ramen, other noodles, gums, dairy products including ice cream, various soups, beverages, tea, drinks, alcoholic beverages, and vitamin complexes, and all health foods in the conventional sense are included.

The mixing amount of the active ingredient may be appropriately determined depending on the purpose of use (prevention, health, or therapeutic treatment). In general, when manufacturing a food or beverage, the composition comprising a lipoteichoic acid of the present invention is added in an amount of 1 to 20 wt %, preferably 5 to 10 wt %, to the raw material. However, in the case of long-term intake for the purpose of health and hygiene or health control, the amount may be below the range, and the active ingredient may be used in an amount above the range because there is no problem in terms of safety. There is no particular limitation on the type of the food. The beverage composition of the invention may contain various flavoring agents or natural carbohydrates as additional ingredients, like a conventional beverage. The natural carbohydrates described above are monosaccharides such as glucose and fructose, disaccharides such as maltose and sucrose, polysaccharides such as dextrin and cyclodextrin, and sugar alcohols such as xylitol, sorbitol and erythritol. As a sweetener, a natural sweetener such as thaumatin and stevia extract, or a synthetic sweetener such as saccharin and aspartame can be used. The proportion of the natural carbohydrate is generally about 0.01 to 0.04 g, preferably about 0.02 to 0.03 g per 100 ml of the composition of the present invention. In addition, the composition of the present invention may contain various nutrients, vitamins, electrolytes, flavoring agents, coloring agents, pectic acid and its salts, alginic acid and its salts, organic acids, protective colloid thickeners, pH regulators, stabilizers, preservatives, glycerin, alcohol, carbonating agents used in carbonated beverages, etc. In addition, the composition of the present invention may contain fruit pulp for the production of natural fruit juice, fruit juice drinks, and vegetable drinks. These components may be used independently or in combination. The proportion of these additives is not particularly important, but is generally selected in the range of 0.01 to 0.1 parts by weight per 100 parts by weight of the composition of the present invention.

The present invention also provides a livestock feed additive comprising the composition.

When used as a feed additive, the composition may be a highly concentrated solution of 20 to 90% or may be manufactured in the form of a powder or granule. The feed additive may additionally contain one or more of organic acids such as citric acid, fumaric acid, adipic acid, lactic acid and malic acid, phosphates such as sodium phosphate, potassium phosphate, acid pyrophosphate, and polyphosphate and natural antioxidants such as polyphenol, catechin, alpha-tocopherol, rosemary extract, vitamin C, green tea extract, licorice extract, chitosan, tannic acid and phytic acid. When used as a feed, the composition may be formulated in the form of a conventional feed and may contain conventional feed ingredients together.

The feed additive and the feed may further include a grain, for example, ground or crushed wheat, oats, barley, corn, and rice; plant-based protein feed, for example, feed mainly composed of rye, soybeans, and sunflower; animal-based protein feed, for example, blood meal, meat meal, bone meal, and fish meal; sugar and dairy products, for example, dry ingredients composed of various types of milk powder and whey powder, and in addition, it may further include nutritional supplements, digestion and absorption enhancers, growth promoters, etc.

The feed additive may be administered to an animal alone or in combination with other feed additives in an edible carrier. In addition, the feed additive may be easily administered to an animal as a top dressing or by mixing them directly into animal feed or in an oral formulation separate from the feed. When the feed additive is administered separately from animal feed, it may be prepared as an immediate-release or sustained-release formulation by combining it with a pharmaceutically acceptable edible carrier, as is well known in the art. These edible carriers may be solid or liquid, for example, corn starch, lactose, sucrose, soybean flakes, peanut oil, olive oil, sesame oil, and propylene glycol. When a solid carrier is used, the feed additive may be a tablet, capsule, powder, troche, or sugar tablet, or a top dressing in a non-dispersible form. When a liquid carrier is used, the feed additive may be a gelatin soft capsule, or a syrup, suspension, emulsion, or solution formulation.

In addition, the feed additive and the feed may contain auxiliary agents, for example, a preservative, a stabilizer, a wetting agent or an emulsifier, a solution accelerator, etc. The feed additive may be used by adding it to the animal feed by injecting, spraying, or mixing it.

The feed or feed additive of the present invention can be applied to various animal diets, including mammals, poultry, and fish.

In the present invention, the animal includes mammals, and the present invention can be applied to mammals, such as pigs, cows, sheep, goats, and experimental rodents, as well as pets (e.g., dogs, cats), and it can be applied to poultry such as chickens, turkeys, ducks, geese, pheasants, and quail, and it can be applied to fish such as trout, etc., but is not limited thereto.

The composition of the present invention can be used as pet snacks, oral care products, hygiene products, and health functional foods for animals, and can be used as pet products without limitation to the above-mentioned examples.

### Advantageous Effects

The lipoteichoic acid according to the present invention inhibits periodontitis and inflammation caused by not only gram-positive bacteria but also gram-negative bacteria, and can be effectively used for preventing or treating of periodontitis. It reduces the bioadhesion force of periodontitis causing bacteria, inhibits biofilm formation, and suppresses infection. Through the use of biocompatible substances, it is possible to increase safety in the human body and reduce side effects.

### DESCRIPTION OF DRAWINGS

FIGS. 1a and 1b show the results of verifying the reduction in Porphyromonas gingivalis attachment force and the therapeutic effects of lipoteichoic acid purified from Lactobacillus reuteri (hereinafter “LreLTA”) using an in vivo model.

FIG. 2 shows the results of verification of the surface attachment force reduction effect of Porphyromonas gingivalis by LreLTA. (a) is the result of analyzing the attachment level of Porphyromonas gingivalis in response to lipoteichoic acid by FACS, (b) is the result of confirming the weakening of the surface attachment force of Porphyromonas gingivalis in a concentration gradient manner, (c) is the result of confirming the weakening of the surface attachment force of the pre-attached Porphyromonas gingivalis in a concentration gradient manner, (d-i) is the result of confirming the efficacy of weakening the attachment force of Porphyromonas gingivalis isolated from an actual periodontitis patient.

FIGS. 3a to 3c are the results of verification that LreLTA inhibits the activity of arginine-specific gingipain, a virulence factor of Porphyromonas gingivalis, and shows the efficacy of alleviating the inflammatory response.

FIGS. 4a to 4d are the results of verification of the association between the fimbrial protein and LreLTA on the attachment force using a fimbriae mutant Porphyromonas gingivalis strain.

FIG. 5 shows the results of comparative analysis of the surface attachment force reduction effect of LTA isolated and purified from various lactic acid bacteria.

FIGS. 6a and 6b show the results of verifying the attachment force reduction effect on Porphyromonas gingivalis and Staphylococcus aureus, respectively, when each functional group was deleted from LreLTA and lipoteichoic acid purified from Lactobacillus plantarum (hereinafter “LpLTA”).

FIG. 7 shows the results of verifying the attachment force reduction effect of LreLTA on other periodontal disease causing bacteria residing in the oral cavity. (a) The results of the attachment force inhibition effect of LreLTA on Campylobacter gracilis, (b) Prevotella nigrescens, (c) Aggregatibacter actinomycetemcomitans, and (d) Parvimonas micra.

FIG. 8 shows the results of the attachment force reduction effect of LreLTA on Gram-negative bacteria residing in other organs. It was confirmed for (a) Escherichia co/i and (b) Pseudomonas aeruginosa, which are pathogenic Gram-negative bacteria residing in the lungs and intestines.

### MODE FOR INVENTION

Hereinafter, the components and effects of the present invention will be described in more detail through examples. These examples are only intended to illustrate the present invention, and the scope of the present invention is not limited by these examples.

### PREPARATION EXAMPLE

### Purification of Lipoteichoic Acid

To purify lipoteichoic acid from Lactobacillus, L. acidophilus KACC 12419, L. casei KCTC 3260, L. rhamnosus GG, L. plantarum KCTC10887 bp, and L. reuteri ATCC 23272 strains were used. Each strain was inoculated into de Man, Rogosa, and Sharpe broth, and the culture was stagnant at 37° C. for 48 h. The bacteria were harvested by centrifugation. The harvested bacteria were homogenized using an ultrasonic grinder, and the homogenate was mixed with an organic solvent, butanol, at an equal ratio, and the lipid layer was separated by centrifugation. Afterwards, the separated extract was sequentially purified using hydrophobic interaction chromatography and anion exchange chromatography to obtain high-purity lipoteichoic acid, and then the organic solvent was completely removed through a dialysis process. The obtained purified product was freeze-dried to obtain the final dried product.

### Example 1

Verification of LreLTA's Attachment Force Reduction and Therapeutic Effect on Porphyromonas gingivalis Through an In Vivo Model

To first verify whether LreLTA has a therapeutic effect in a periodontitis-induced model caused by Porphyromonas gingivalis (ATCC 33277), the Porphyromonas gingivalis strain was used, and wild-type mice of the C57BL/6 strain were purchased from Dooyeol Biotech as experimental animals for the in vivo model, and animal experiments were performed in strict compliance with the ethical regulations for experimental animal research.

In a dental ligation animal model in which periodontitis was induced by tying the mouse maxillary second molars with a 5-0 suture, the mice were divided into a total of 5 groups. One group was not administered anything or ligated (control group), one group was ligated without administering anything (dental ligation, negative control group), one group was administered 50 g of LreLTA alone to the ligation site for 7 days (dental ligation+lipoteichoic acid), one group was injected with 1×10⁹ CFU of Porphyromonas gingivalis strain cultured in a medium supplemented with hemin and menadione in Brain-Heart infusion (BHI) every other day for a total of 3 times to aggravate periodontitis (dental ligation+Porphyromonas gingivalis, positive control group), and finally, the remaining one group was injected with Porphyromonas gingivalis to the ligation site every other day for 3 days under the same conditions, simultaneously with the administration of LreLTA 50 g alone for 7 days (dental ligation+Porphyromonas gingivalis+lipoteichoic acid).

TABLE 1

| Group | Treatment method |
|---|---|
| control group | Nothing administered or ligation. |
| dental ligation, (negative | Ligation without administration. |
| control group) | |
| dental ligation + lipoteichoic | LreLTA 50 μg administered alone to the ligation site for 7 |
| acid | days. |
| dental ligation + | Porphyromonas gingivalis strain 1 × 10⁹ CFU cultured in a |
| porphyromonas gingivalis, | medium supplied with hemin and menadione in Brain-Heart |
| (positive control group) | infusion (BHI) was injected into the ligation site every other |
| | day for 3 days to aggravate periodontitis. |
| dental ligation + | Porphyromonas gingivalis was administered every other day |
| porphyromonas gingivalis + | for 3 days under the same conditions, and LreLTA 50 μg |
| lipoteichoic acid | administered alone for 7 days. |

After the 8th day of administration, the mice were sacrificed, the maxilla was separated to obtain the molars, and the degree of bone destruction was compared through Micro-CT analysis. In order to confirm the improvement of periodontitis, the distance between the alveolar bone at the Cementoenamel Junction-Alveolar Bone Crest (CEJ-ABC) of the tooth was measured after Micro-CT analysis to determine the degree of gap between the teeth and gums.

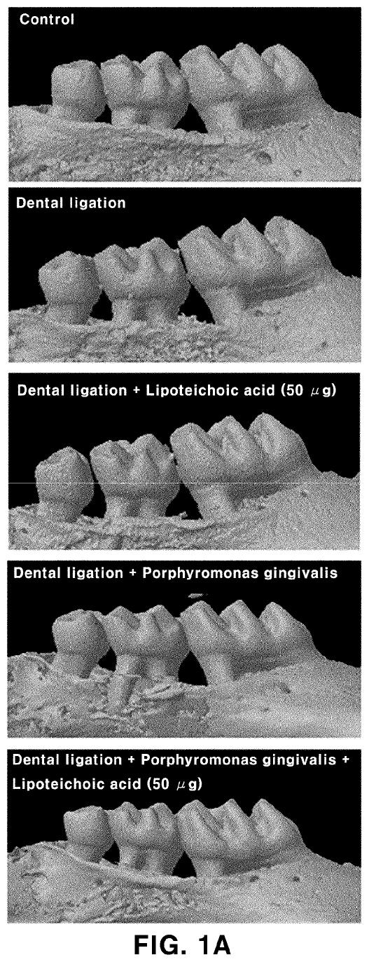

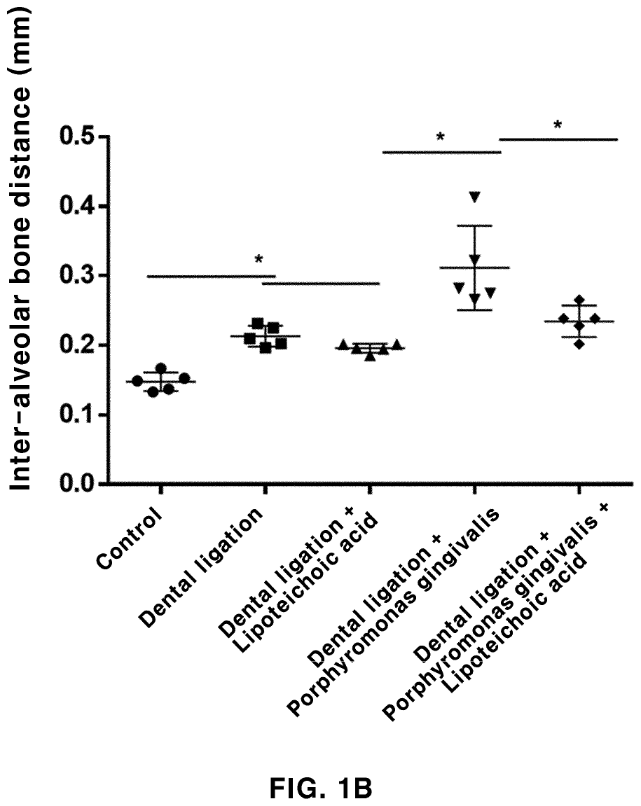

As a result of the experiment, the group treated with Porphyromonas gingivalis and LreLTA showed a higher level of alleviation of periodontitis aggravated by Porphyromonas gingivalis compared to the positive control group treated only with Porphyromonas gingivalis. Through this, it was confirmed that LreLTA can alleviate the induced periodontitis by reducing the attachment force of Porphyromonas gingivalis in living tissue (FIGS. 1a, b).

### Example 2

Verification of the Effect of LreLTA on Reducing the Surface Attachment Force of Porphyromonas gingivalis

To further verify whether LreLTA can adhere to Porphyromonas gingivalis, we first confirmed whether LreLTA directly binds to Porphyromonas gingivalis.

After adding FITC to LreLTA and conjugating it, unconjugated FITC was removed using a PD-10 column, and pure FITC-conjugated LreLTA was quantified and stirred with the wild-type strain Porphyromonas gingivalis for one hour to perform FACS analysis.

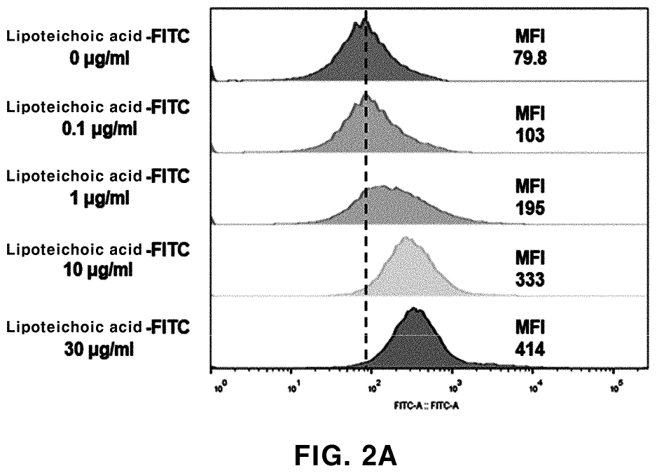

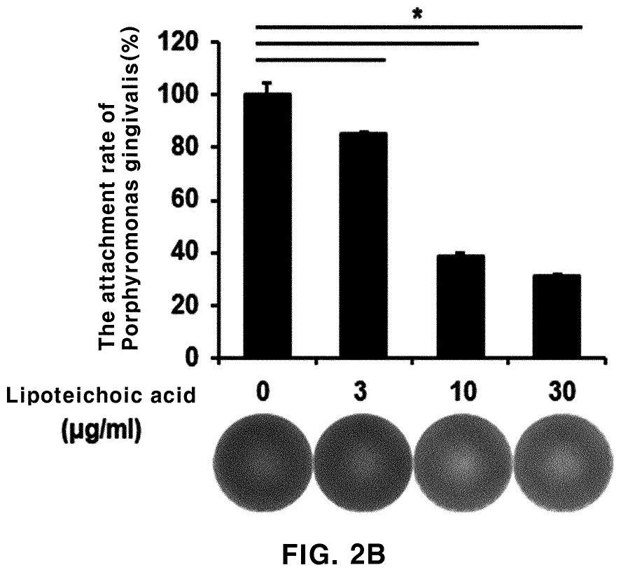

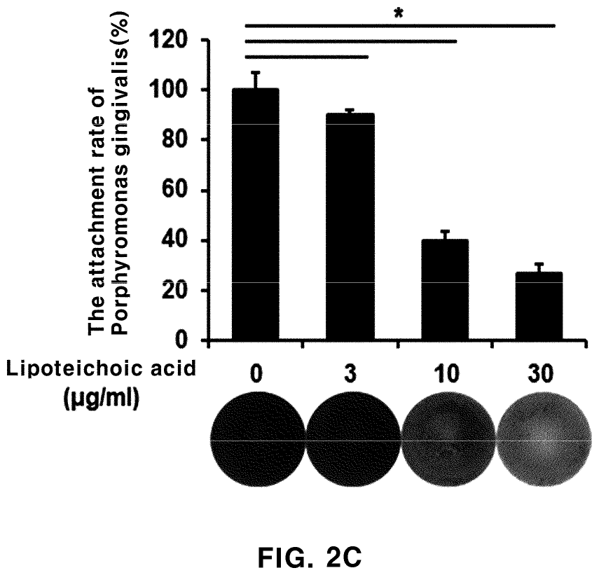

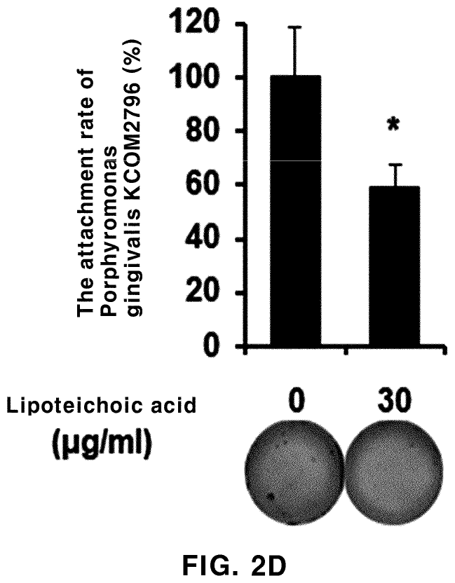

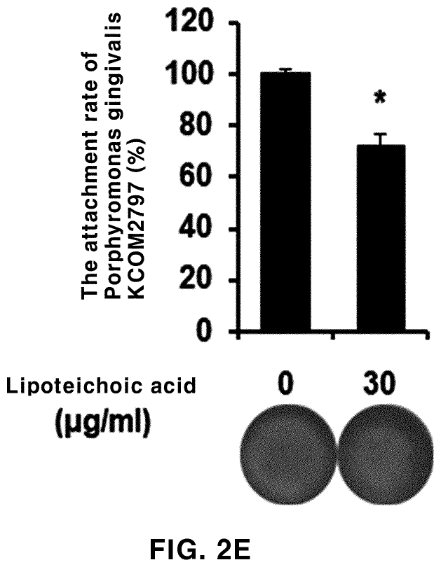

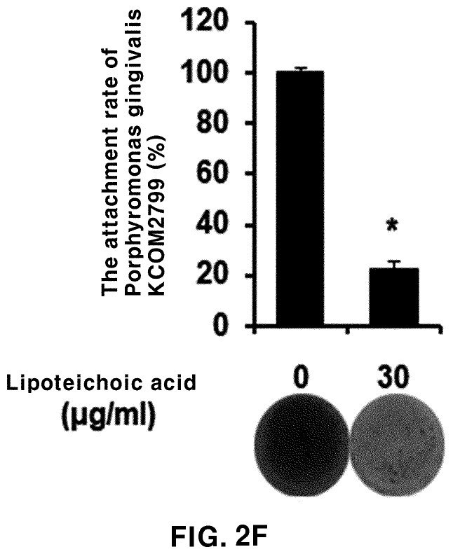

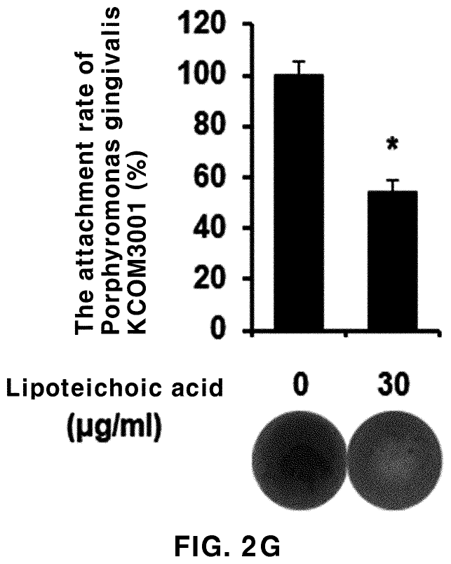

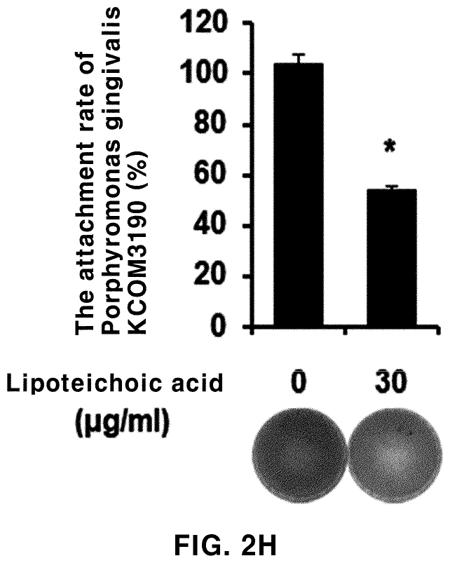

As a result of the experiment, it was confirmed that LreLTA attaches to the surface of Porphyromonas gingivalis in a concentration gradient and exhibits fluorescence (FIG. 2a).

To verify the effect of LreLTA on the surface attachment force of Porphyromonas gingivalis, the wild-type strain Porphyromonas gingivalis was used.

Porphyromonas gingivalis was cultured in BHI medium supplemented with hemin and menadione, and then Porphyromonas gingivalis was prepared at 1×10⁹ CFU/ml and treated with LreLTA at concentrations of 3, 10, and 30 μg/ml. After culturing for 72 h at 37° C. under anaerobic conditions, the degree of surface attachment maintenance was compared using 0.1% crystal violet staining.

As a result of the experiment, it was confirmed that LreLTA reduced the surface attachment force of Porphyromonas gingivalis in a concentration gradient manner (FIG. 2b).

To determine whether the effect of LreLTA in reducing the attachment effect of Porphyromonas gingivalis is the same in the case of Porphyromonas gingivalis that has been attached, Porphyromonas gingivalis strains were cultured in a BHI medium supplied with hemin, and menadione, prepared at 1×10⁹ CFU/ml, and attached at 37° C. under anaerobic conditions for 72 h. After that, LreLTA was treated at concentrations of 3, 10, and 30 μg/ml to the attached surface, and cultured at 37° C. under anaerobic conditions for 24 h. The degree of surface attachment maintenance was compared using 0.1% crystal violet staining.

As a result of the experiment, it was confirmed that LreLTA showed a decrease in the surface attachment force of Porphyromonas gingivalis in a concentration gradient manner (FIG. 2c).

In addition, to confirm whether LreLTA has the same effect on Porphyromonas gingivalis strains isolated from actual periodontitis patients in addition to the wild-type strain, Porphyromonas gingivalis strains KCOM 2796, KCOM 2797, KCOM 2799, KCOM 3001, KCOM 3190, and KCOM 3153 were cultured in BHI medium supplemented with hemin and menadione, and Porphyromonas gingivalis was prepared at 1×10⁹ CFU/ml and treated with LreLTA at a single concentration of 30 μg/ml. After culturing for 72 h at 37° C. under anaerobic conditions, the degree of surface attachment maintenance was compared using 0.1% crystal violet staining.

As a result of the experiment, it was confirmed that LreLTA also reduced the surface attachment force of clinical Porphyromonas gingivalis isolates (FIGS. 2d-i).

### Example 3

Verification of the Efficacy of LreLTA in Alleviating the Inflammatory Response of Porphyromonas gingivalis

The cause of periodontal disease caused by Porphyromonas gingivalis is a chronic inflammatory response. This is caused by arginine-specific gingipain, a toxic factor that induces cell surface attachment and maturation of cell surface attachment factors and degrades MCPIP-1, an immune regulatory factor that suppresses excessive immune responses.

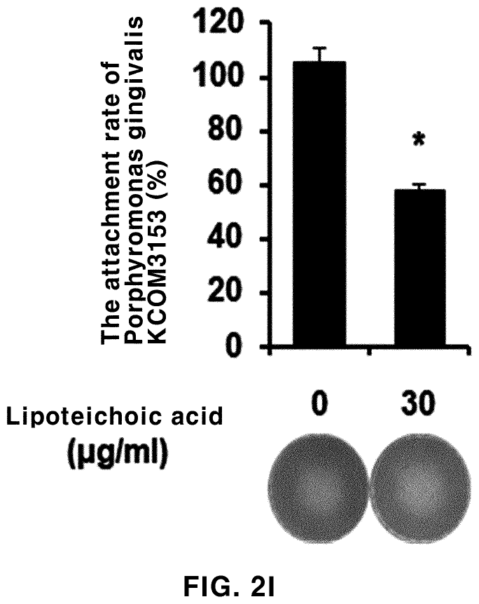

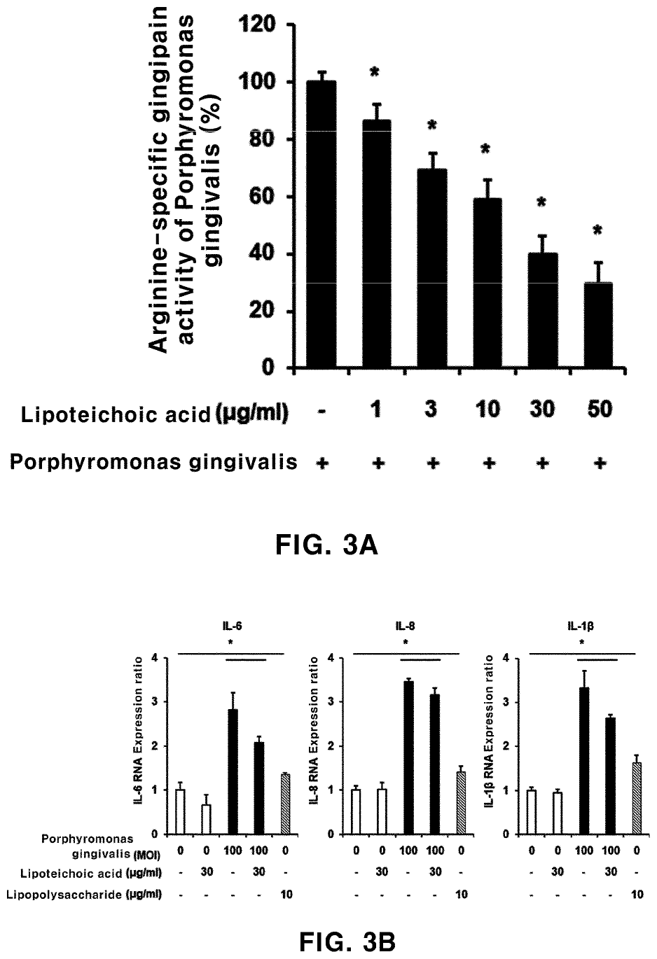

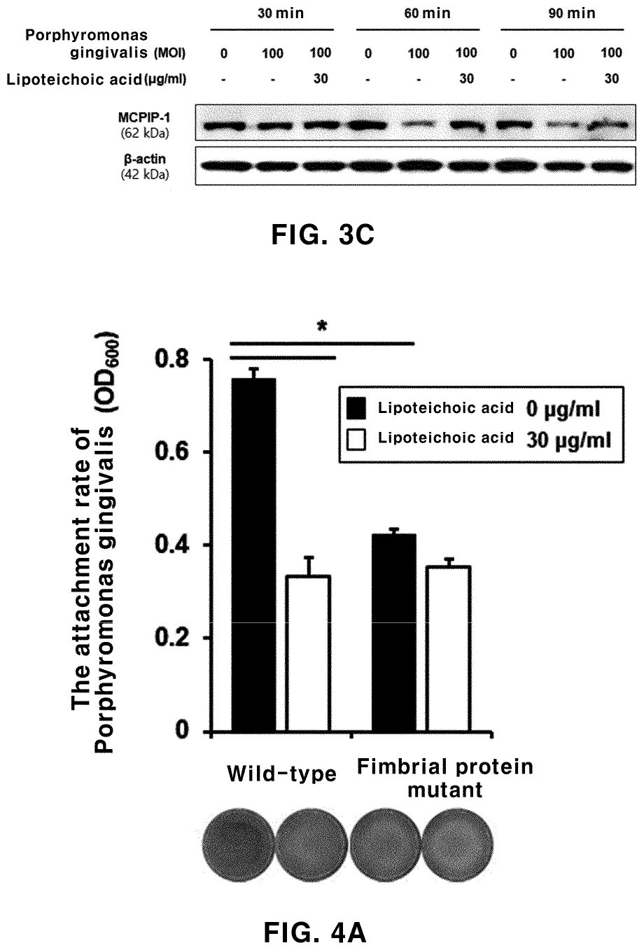

First, through experiments using substrate-specific reactions, it was confirmed that LreLTA can effectively inhibit the activity of arginine-specific gingipain (FIG. 3a).

To confirm whether LreLTA can effectively alleviate the immune response induced by Porphyromonas gingivalis, oral epithelial cells, YD-38 cells (5×10⁵ cells/ml), were infected with Porphyromonas gingivalis at a 100-fold higher level (MOI 100), and the RNA expression of pro-inflammatory cytokines IL-6, IL-8, and IL-1β was analyzed using real-time PCR under conditions in which LreLTA was treated with or without 30 μg/ml.

The experimental results confirmed that LreLTA can suppress the expression of pro-inflammatory cytokines induced by Porphyromonas gingivalis (FIG. 3b).

Additionally, the protein expression of MCPIP-1, which controls the inhibition of the pro-inflammatory cytokines, was confirmed through Western blotting under the above-mentioned method conditions, and it was confirmed that LreLTA effectively inhibits the activity of arginine-specific gingipain of Porphyromonas gingivalis, thereby ultimately inhibiting the degradation of MCPIP-1 and alleviating the inflammatory response (FIG. 3c).

### Example 4

Verification of the Association Between LreLTA and Porphyromonas gingivalis Fimbriae Mutant Strains

According to previous studies, FimA, a fimbrial protein, is known to act as a virulence factor of Porphyromonas gingivalis and is responsible for cell attachment and invasion. Therefore, the present inventors created a fimbrial protein mutant strain for the strain and conducted research. The method was performed under the same conditions as Example 2.

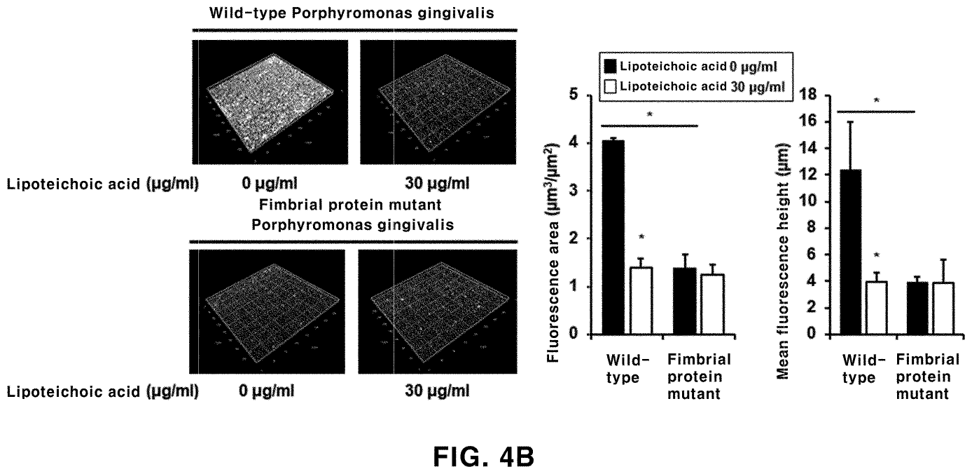

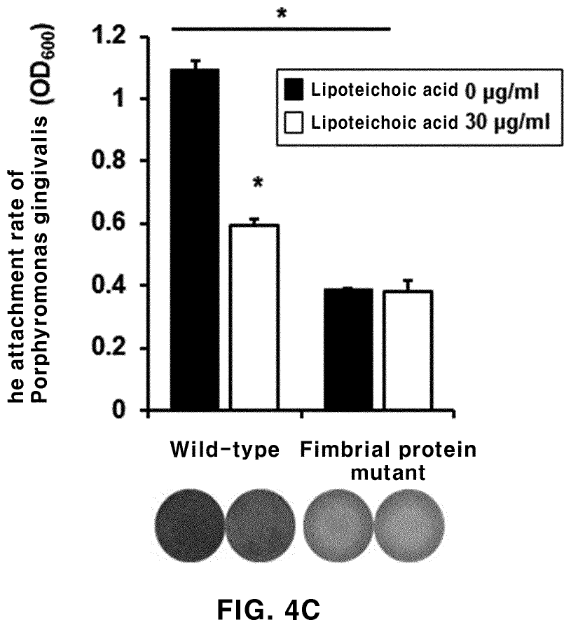

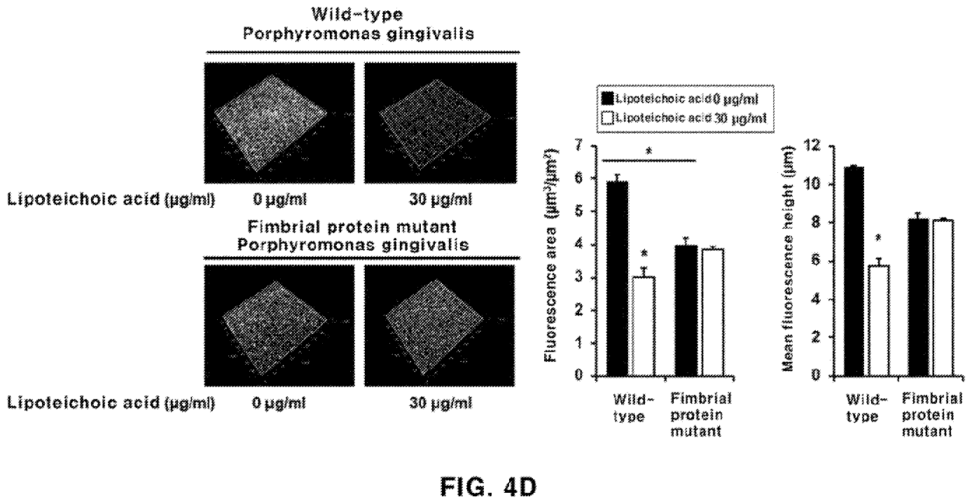

As a result of the experiment, it was confirmed that the surface attachment force of Porphyromonas gingivalis was not reduced by LreLTA in the fimbriae mutant strain under a single concentration treatment condition, and it was confirmed in the attachment process and the pre-attachment situation. In addition, this was further verified through the confocal microscopy results under the same experimental conditions, and it was clarified that LreLTA directly targets fimbrial protein and inhibits attachment (FIGS. 4a-d).

### Example 5

Effect of LTA Isolated and Purified from Various Lactic Acid Bacteria on the Surface Attachment Force of Porphyromonas gingivalis

In order to determine the effect of LTA isolated and purified from various lactic acid bacteria on the surface attachment force of Porphyromonas gingivalis, LTA isolated and purified from five types of lactic acid bacteria were used.

Porphyromonas gingivalis strains were cultured in a BHI medium supplied with hemin, and menadione, and the cell count was prepared at 1×10⁹ CFU/ml, and treated with five types of LTAs at a single concentration of 30 μg/ml. After culturing for 72 hours at 37° C. under anaerobic conditions, the degree of surface attachment maintenance was compared using 0.1% crystal violet staining.

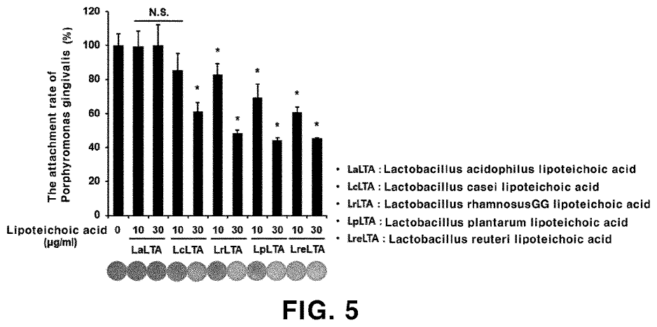

Experimental results showed that all five types of LTAs except LaLTA reduced the surface attachment force of Porphyromonas gingivalis, and among the LTAs isolated from human beneficial lactic acid bacteria, LreLTA in particular reduced the surface attachment force of Porphyromonas gingivalis most effectively (FIG. 5a, LaLTA: Lactobacillus acidophilus lipoteichoic acid; LrLTA: Lactobacillus rhamnosus lipoteichoic acid; LpLTA: Lactobacillus plantarum lipoteichoic acid; LcLTA: Lactobacillus casei lipoteichoic acid; LreLTA: Lactobacillus reuteri lipoteichoic acid).

### Example 6

### Verification of the Effect of Reducing Surface Attachment Force Through the LTA Functional Group Deletion Experiment of Lactic Acid Bacteria

When the functional group of LreLTA was deleted, the effect on the attachment force of Porphyromonas gingivalis was confirmed, and the difference from the existing reports was confirmed.

In order to delete D-alanine, which gives positive charge to LTA of lactic acid bacteria, Tris-HCl pH 8.5 aqueous solution that can break D-alanine bonds was used, stirred with LTA for 24 hours, dialyzed for 48 hours, and then LTA lacking D-alanine was quantified and used in the experiment. In order to delete both lipids involved in hydrophobic interactions and D-alanine that gives positive charge to LTA of lactic acid bacteria, 0.5N NaOH, an alkaline solution at pH 12, was used, stirred with LTA for 2 hours, dialyzed and quantified in the same manner as above, and used in the experiment. The lack of functional groups in the LTA to be used in the experiment was confirmed using a 1% ninhydrin solution for detecting amino acids and a 5% phosphomolybdic acid solution for detecting lipids. After confirming the lack of functional groups, Porphyromonas gingivalis strains were cultured in a BHI medium supplied with hemin and menadione, and the cell number was prepared at 1×10⁹ CFU/ml, and LTA with or without the lack of functional groups was treated at a single concentration of 30 μg/ml. After cultured for 72 h at 37° C. under anaerobic conditions, the degree of surface attachment maintenance was confirmed through 0.1% crystal violet staining. In addition, in order to compare and analyze the results of LTA lacking functional groups for Staphylococcus aureus RN4220, which was used in existing reports, the cell number was prepared at 1×10⁹ CFU/ml in Luria-Bertani (LB) medium and treated with LTA with or without the lack of functional groups at a single concentration of 30 μg/ml. After culturing for 24 hours at 37° C. under aerobic conditions, the degree of surface attachment maintenance was compared through 0.1% crystal violet staining.

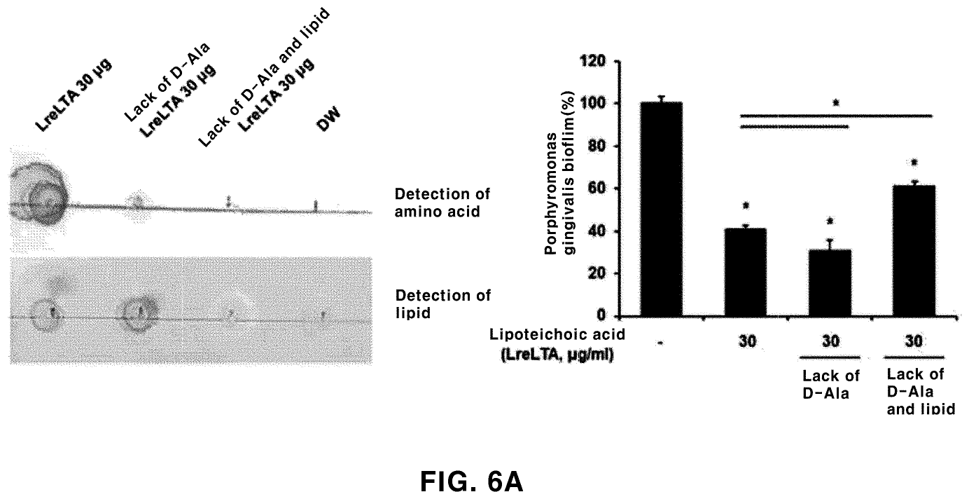

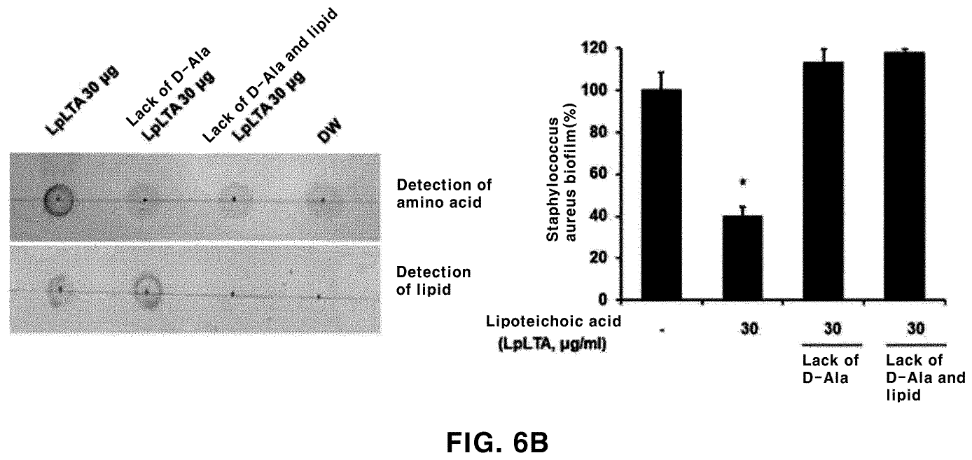

Experimental results showed that in the case of LreLTA, when D-alanine was lacking, the inhibition of Porphyromonas gingivalis attachment force, a gram-negative bacterium, was more effective than that of LTA without lack in inhibiting Porphyromonas gingivalis attachment force (FIG. 6a). However, LpLTA, which was used in existing reports, showed no effect on the attachment force of Staphylococcus aureus, a gram-positive bacterium, when the functional group was lacking (FIG. 6b). These results confirm that Lactobacillus LTA lacking the functional group has differences in attachment force control depending on whether it is a gram-positive or -negative bacterium.

### Example 7

### Verification of the Effect of Reducing Attachment Force to Other Periodontitis Causing Bacteria Residing in the Oral Cavity

In reality, the mechanism of periodontitis in the oral cavity is generally mainly caused by Gram-negative bacteria, and not only Porphyromonas gingivalis but also various Gram-negative bacteria are closely related to periodontitis. Therefore, the effect of Lactobacillus LTA on other Gram-negative bacteria that cause periodontitis was additionally confirmed.

Using various media, Campylobacter gracilis, Prevotella nigrescens, and Aggregatibacter actinomycetemcomitans, which are Gram-negative bacteria associated with periodontitis, were treated with LTA at a single concentration of 30 μg/ml, and then cultured at 37° C. under anaerobic conditions. The degree of surface attachment maintenance was compared using 0.1% crystal violet staining. Parvimonas micra, one of the Gram-positive bacteria closely associated with periodontitis, was also compared for its attachment ability to LTA.

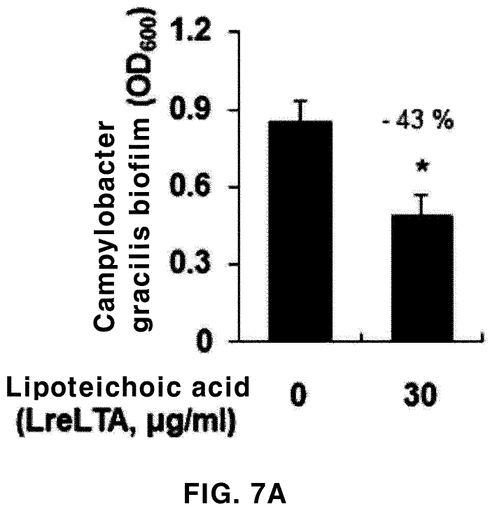

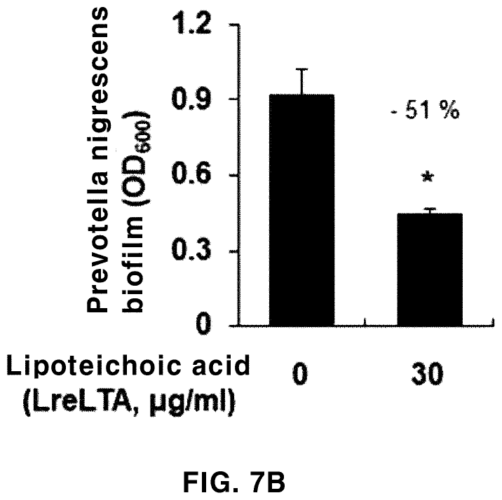

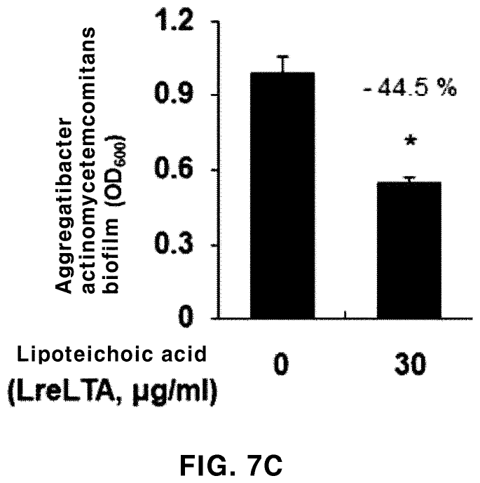

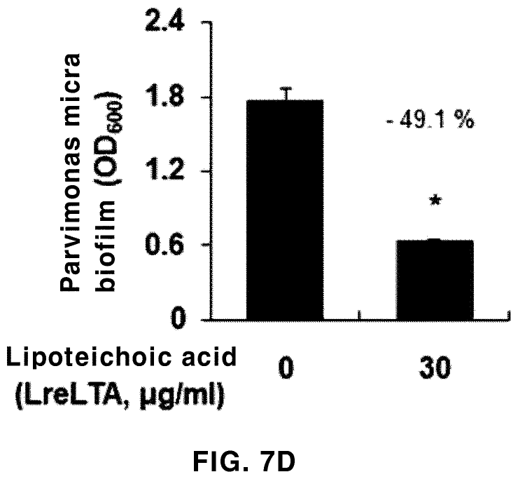

The experimental results confirmed that LreLTA not only effectively reduces the attachment force of Gram-negative bacteria closely associated with periodontitis, but also reduces the attachment force of Gram-positive bacteria (FIGS. 7a-d).

Through these results, it was confirmed that Lactobacillus LTA can reduce the attachment force of Gram-positive bacteria, as well as the attachment force of Gram-negative bacteria with structural differences, in addition to previous studies that showed that it can reduce the attachment force of Gram-positive bacteria.

### Example 8

### Verification of the Attachment Force Reduction Effect on Gram-Negative Bacteria Residing in Other Organs

Based on the above results that LreLTA can effectively inhibit the attachment of bacteria residing in the oral cavity, the effect on the attachment of Gram-negative bacteria residing in other organs was additionally confirmed.

Using LB medium, Escherichia coli and Pseudomonas aeruginosa, pathogenic Gram-negative bacteria residing in the lungs and intestines, were treated with LreLTA at a single concentration of 30 μg/ml, and then cultured at 37° C. under aerobic conditions, and the degree of surface adhesion maintenance was compared through 0.1% crystal violet staining.

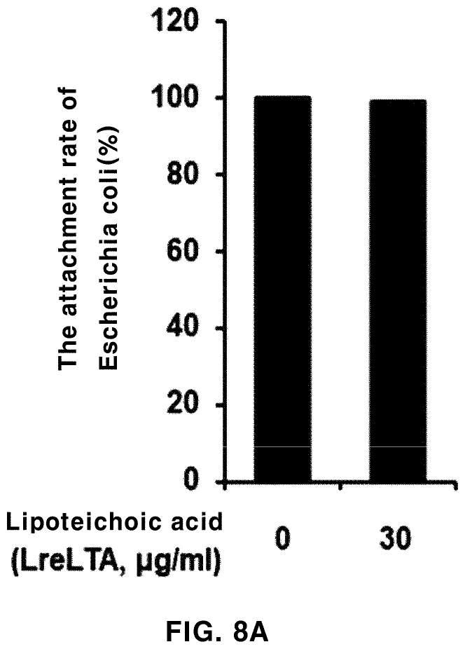

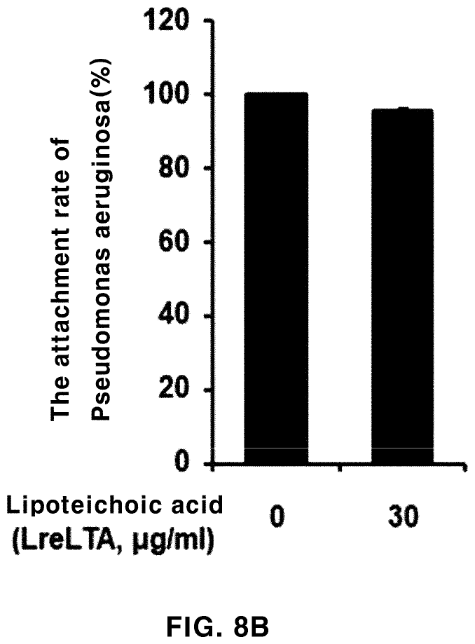

The experimental results confirmed that LreLTA did not affect the attachment of Gram-negative bacteria residing in other organs (FIG. 8a, b). These results confirm that, in addition to previous studies showing that Lactobacillus LTA can reduce the attachment force of Gram-positive bacteria, it can also reduce the attachment force of Gram-negative bacteria that are structurally different from Gram-positive bacteria, but it shows a reducing effect on Gram-negative bacteria residing in the oral cavity.

1. A pharmaceutical composition for preventing or treating periodontitis, comprising lipoteichoic acid or a pharmaceutically acceptable salt thereof.

2. The pharmaceutical composition of claim 1,
wherein the lipoteichoic acid is isolated from at least one selected from a group consisting of Lactobacillus rhamnosus, Lactobacillus plantarum, Lactobacillus casei and Lactobacillus reuteri.

3. The pharmaceutical composition of claim 1,
wherein the periodontitis causing bacteria is at least one selected from a group consisting of Porphyromonas gingivalis, Campylobacter gracilis, Prevotella nigrescens, Aggregatibacter actinomycetemcomitans and Parvimonas micra.

4. The pharmaceutical composition of claim 3,
wherein the lipoteichoic acid targets fimbrial protein FimA when the periodontitis causing bacteria is Porphyromonas gingivalis.

5. An anti-inflammatory composition, comprising lipoteichoic acid or a pharmaceutically acceptable salt thereof.

6. The anti-inflammatory composition of claim 5,
wherein the lipoteichoic acid is isolated from at least one selected from a group consisting of Lactobacillus rhamnosus, Lactobacillus plantarum, Lactobacillus casei and Lactobacillus reuteri.

7. The anti-inflammatory composition of claim 5,
wherein the inflammation is caused by Porphyromonas gingivalis.

8. The anti-inflammatory composition of claim 5,
wherein the composition alleviates inflammatory responses by inhibiting degradation of MCPIP-1.

9-10. (canceled)

11. A health functional food, comprising the composition of claim 1.

12. A livestock feed additive, comprising the composition-any of claim 1.
